(19) United States
(12) Patent Application Publication
(10) Pub. No.: US 2007/0142872 A1
(43) Pub. Date: Jun. 21, 2007

(54) Deep brain stimulation apparatus, and associated methods

(76) Inventors: Marlin H. Mickle, Pittsburgh, PA (US); Steven A. Hackworth, Pittsburgh, PA (US)

Correspondence Address: ECKERT SEAMANS CHERIN & MELLOTT, 600 GRANT STREET, 44TH FLOOR, PITTSBURGH, PA, 15219, US

(21) Appl. No.: 11/313,459

(22) Filed: Dec. 21, 2005

(51) Int. Cl. A61N 1/00

(52) U.S. Cl. 607/45

(57) ABSTRACT

Various methods and apparatus for providing deep brain stimulation for the treatment of diseases such as Parkinson's Disease that do not require an onboard power supply that is implanted in the patient's body. Power may be supplied from outside of the body by, for example, near-field inductive coupling with an external power supply provided in, for example, a headgear worn by the patient. Power may also be supplied by providing an antenna for harvesting ambient energy, such as ambient RF energy, and converting it into DC power. In addition, the methods and apparatus provide for remote, wireless programming of the parameters that specify the nature of electrical pulses provided to the brain via probes implanted in the brain.

### CROSS-REFERENCE TO RELATED APPLICATION

This application is a continuation-in-part of U.S. application Ser. No. ______, entitled “Deep Brain Stimulation Apparatus, And Associated Method,” filed on Dec. 20, 2005, which claims the benefit of U.S. Provisional Application No. 60/638,037, entitled “Deep Brain Stimulation,” which was filed on Dec. 21, 2004, the disclosure of which is incorporated herein by reference.

### GOVERNMENT CONTRACT

This work was supported in part by a grant from the National Science Foundation under Contract No. EEC 0203341. The United States government may have certain rights in the invention described herein.

### FIELD OF THE INVENTION

The present invention relates to methods and apparatus for providing treatment for the symptoms of various diseases, such as Parkinson's Disease (tremors), and in particular to improved methods and apparatus for providing deep brain electrical stimulation.

### BACKGROUND OF THE INVENTION

Parkinson's Disease is a neurodegenerative disorder that causes muscular tremors, stiffness, and slowness of movement. The first line of treatment for Parkinson's is the administration of drugs. Over a period of time, these drugs slowly lose their effect to arrest the symptoms associated with Parkinson's. Once a patient enters a refractory stage of the disease in which drugs are not effective, one alternative treatment option to reduce associated tremors is Deep Brain Stimulation (DBS). DBS can also be used as a part of a treatment plan for other diseases, such as Huntington's disease, dystonia, and epilepsy, among others.

In DBS, one or more probes are implanted in the basal ganglia area of the brain to administer electric pulses that curb Parkinson's symptoms (or the symptoms of the other diseases mentioned above). Although not fully understood, DBS is becoming a more and more widely accepted treatment, with various implantable devices currently being on the market. An example of such a device is the Active Therapy System sold by Medtronic, Inc. of Minneapolis, Minn. (www.medtronic.com/physician/activa/implantable.html). These devices, however, require the implantation of a relatively large battery and control pack in the chest with subcutaneous wires threaded up through the neck to the top of the skull and ultimately to the implanted probes (one or more). The control pack and wires are a common source of irritation and infection, sometimes necessitating long periods of antibiotics or even removal of the device. Furthermore, such devices are susceptible to a limited battery life and magnetic interference. After the average 3- to 5-year lifespan of an implant's battery, another surgery is required to replace the device. Thus, it would be advantageous to be able to provide DBS in a manner that eliminates the intrusive battery pack and wires, as well as the health risks commonly associated with them.

### SUMMARY OF THE INVENTION

The present invention relates to an apparatus for providing electrical stimulation to the brain of a patient for treating, for example, Parkinson's disease. The apparatus includes one or more probes for being implanted in the patient's brain and for providing electrical pulses to the brain. The apparatus also includes an implantable device for being implanted subcutaneously in the patient's head that has: (i) control circuitry adapted to generate the electrical pulses and provide the electrical pulses to the probes, and (ii) power circuitry for providing a DC power signal to the control circuitry. A power supply separate from the implantable device provided at a stationary location separate from the implantable device and external to the patient's body is also provided. The power supply provides power to the implantable device through a near-field technique, such as near-field inductive coupling, between the power supply and the power circuitry when the power circuitry is in proximity with the power supply. In particular, the power supply preferably includes an oscillator and a primary winding, wherein the oscillator generates a first AC signal and provides the first AC signal to the primary winding. The power circuitry includes a secondary winding, and the first AC signal induces a second AC signal in the secondary winding when the secondary winding is in proximity with the primary winding. The power circuitry converts the second AC signal into the DC power signal. The power circuitry also preferably includes an energy storage device for storing energy for subsequent use by the implantable device, particularly when the implantable device is located a certain distance from the power supply.

The control circuitry preferably includes a programmable processor and a wireless communications device. The programmable processor controls the generation of the electrical pulses based upon one or more pulse parameters. In this embodiment, the apparatus further includes a remote programming device external to the patient's body that is adapted to wirelessly transmit programming signals to the wireless communications device which are then provided to the programmable processor for adjusting the one or more pulse parameters. The one of more pulse parameters may specify one or more of a frequency of the electrical pulses, an amplitude of the electrical pulses, a pulse width of the electrical pulses, an on/off state of the electrical pulses, and an application location (i.e., to which electrodes) of the electrical pulses.

A method of providing electrical stimulation to the brain of a patient is also provided that includes steps of implanting one or more probes into the brain, implanting a device subcutaneously in the patient's head, causing the device to generate electrical pulses and provide the electrical pulses to the one or more probes, and providing power to the device from a stationary location external to the patient's body using a near-field technique, such as near-field inductive coupling. The method may further include selectively wirelessly adjusting the one or more pulse parameters from a second location external to the patient's body.

In another embodiment, the invention relates to a method of treating a neurodegenerative disease, such as Parkinson's Disease, including steps of implanting a device in the head of a patient, causing the device to generate and provide electrical pulses to the brain, and providing power to the device from a stationary location separate from the device and external to the patient's body using a near field technique such as near-field inductive coupling.

It is an object of this invention to provide a method and apparatus for providing deep brain stimulation that does not require an onboard power supply that is implanted within the body of the patient.

It is a further object of this invention to provide a method and apparatus for providing deep brain stimulation that eliminates the problems associated with the subcutaneous wires that are associated with prior art devices.

It is still a further object of this invention to provide a method and apparatus for providing deep brain stimulation that eliminates the battery life and replacement problems associated with prior art devices.

It is still a further object of this invention to provide a method and apparatus for providing deep brain stimulation that is powered by a near-field technique, such as near-field inductive coupling.

It is still a further object of this invention to provide a method and apparatus for providing deep brain stimulation that allows the electrical pulse parameters to be readily and non-intrusively adjusted from outside of the body.

It is still a further object of this invention to provide a method of treating a neurodegenerative disease such as Parkinson's Disease.

### BRIEF DESCRIPTION OF THE DRAWINGS

The accompanying drawings illustrate presently preferred embodiments of the invention, and together with the general description given above and the detailed description given below, serve to explain the principles of the invention. As shown throughout the drawings, like reference numerals designate like or corresponding parts.

FIG. 1 is a block diagram of a DBS device according to a first embodiment of the present invention;

FIG. 2 is a block diagram of control circuitry for driving the probes of the DBS device of FIG. 1 according to one embodiment of the invention;

FIG. 3 is a schematic illustration of the parameters used to specify the electrical pulses used in the present invention;

FIG. 4 is a block diagram of a remote programming device that allows an operator to set pulsing parameters for the DBS devices described herein;

FIG. 5 is a block diagram of an implantable DBS device according to an alternative embodiment of the present invention; and

FIG. 6 is a block diagram of a DBS device according to a further alternative embodiment of the present invention.

### DESCRIPTION OF THE PREFERRED EMBODIMENTS

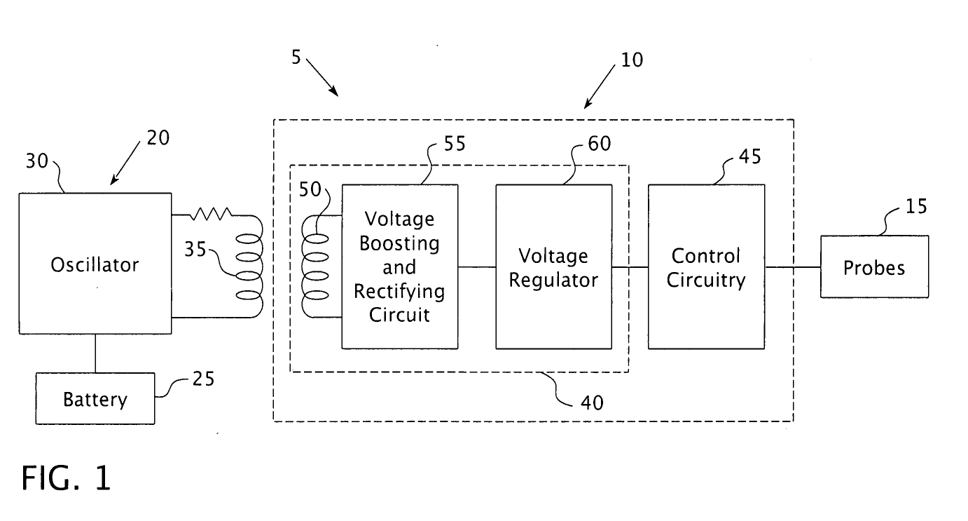

FIG. 1 is a block diagram of a DBS device 5 according to a first embodiment of the present invention for use in providing treatment to a patient, which preferably is a human, but may even include an animal. The DBS device 5 includes an implantable device 10 that is implanted subcutaneously in the head, i.e., it is mounted on the skull and below the skin. As such, the implantable device 10 may be implanted using only a local anesthetic. In addition, because the implantable device 10 is implanted in the body, the components thereof are provided on some type of biologically compatible substrate and encased in some type of biologically compatible material, such as a substrate or housing made from an accepted medical polymer. As described in greater detail herein, the implantable device 10 controls and drives one or more probes 15 which are implanted in the basal ganglia area of the brain by generating and providing to the probes 15 appropriate electrical pulses. The probes 15, in turn, administer the electrical pulses to the brain. Typically, each probe 15 is an elongated member that includes one or more electrodes along its length for actually applying the pulses to the brain. Because the electrodes are provided along the length of a probe 15, the electrical pulses can be provided at different depths within the brain.

As will be appreciated, the electronic components of the implantable device 10 require power in order to operate. The implantable device 10 does not, however, have an onboard power supply such as a battery. Instead, the embodiment of the implantable device 10 shown in FIG. 1 is remotely powered using a near-field technique, which in the embodiment shown in FIG. 1 is near-field inductive coupling. The definition of the near-field is generally accepted as a region that is in proximity to an antenna or another radiating structure where the electric and magnetic fields do not have a plane-wave characteristic but vary greatly from one point to another. Furthermore, the near-field can be subdivided into two regions which are named the reactive near field and the radiating near field. The reactive near-field is closest to the radiating antenna and contains almost all of the stored energy, whereas the radiating near-field is where the radiation field is dominant over the reactive field but does not posses plane-wave characteristics and is complicated in structure. This is in contrast to the far-field, which is generally defined as the region where the electromagnetic field has a plane-wave characteristic, i.e. it has a uniform distribution of the electric and magnetic field strength in planes transverse to the direction of propagation.

In particular, in an embodiment where near-field inductive coupling is used, the DBS device 5 includes a separate, external power supply 20 that is, in one particular embodiment, provided in headgear, such as a hat or cap, worn by the patient. The power supply 20 includes a battery 25 that is electrically connected to an adjustable oscillator 30 which generates an AC signal. A suitable example of an oscillator that may be used for the oscillator 30 is the LTC6900 precision low power oscillator sold by Linear Technology Corporation of Milpitas, Calif., which is capable of generating 50% duty cycle square waves at frequencies of between 1 KHz and 20 MHz. Other types/shapes of waveforms and/or duty cycles may also be used. The power supply also includes a primary winding 35 that is electrically connected to the oscillator 30 and receives the waveform generated thereby.

The implantable device 10 is provided with power circuitry 40 that provides a DC signal of an appropriate level for powering the control circuitry 45 provided as part of the implantable device 10. As described in greater detail herein, the control circuitry 45 controls the generation of the electrical pulses provided to the probes 15 (and ultimately to the patient's brain). As seen in FIG. 1, the power circuitry 40 includes a secondary winding 50, a voltage boosting and rectifying circuit 55 and a voltage regulator 60. In operation, when the AC signal is provided to the primary winding 35, a second AC signal is induced in the secondary winding 50 as a result of near-field inductive coupling with the primary winding 35.

Because of losses that occur in the inductive coupling, it is preferred to increase the voltage of the induced AC signal in order to provide a supply voltage of an appropriate level to the control circuitry 45 (as described hereinafter, the highest voltage necessary for the control circuitry 45 is typically 3 V, and the required voltage ranges from 1.5 V to 3 V, although voltages to 5 V may also be desired). In addition, because a DC signal is employed to power the control circuitry 45, the induced AC signal is also converted to DC. Thus, the induced AC signal is provided to the voltage boosting and rectifying circuit 55, which increases the voltage of and rectifies the received AC signal. In one particular embodiment, the voltage boosting and rectifying circuit 55 is a one or more stage charge pump, sometimes referred to as a “voltage multiplier.” Charge pumps are well known in the art. Basically, one stage of a charge pump essentially doubles the amplitude of an AC input voltage and stores the doubled DC voltage on an output capacitor. The voltage could also be stored using a rechargeable battery. Successive stages of a charge pump, if present, will essentially double the voltage from the previous stage. The DC signal that is output by the voltage boosting and rectifying circuit 55 is provided to a voltage regulator 60, which in turn provides a regulated DC voltage signal to the control circuitry 45. The voltage regulator 60 is primarily provided to resist spikes in the DC voltage signal provided to the control circuitry 45 and to resist DC voltage signals that may overdrive the control circuitry 45.

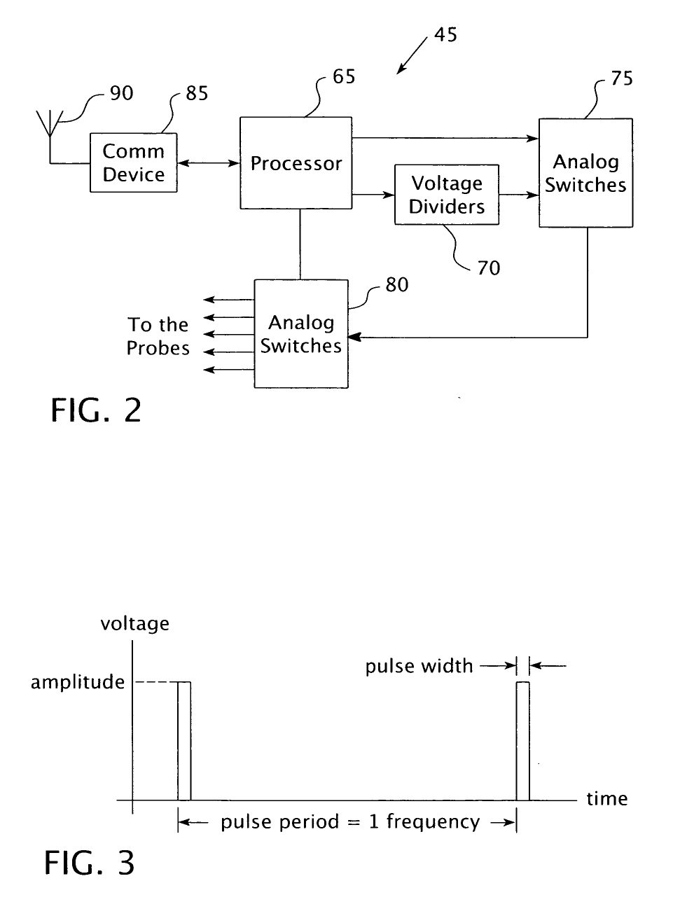

FIG. 2 is a block diagram of the control circuitry 45 for driving the probes 15 according to one embodiment of the invention. The control circuitry 45 includes a processor 65, such as a microcontroller or some other type of microprocessor. A suitable example of the processor 65 is the PIC16LF87 microcontroller sold by Microchip technology, Inc. of Chandler, Ariz. The processor 65 is programmed to output the actual pulses to be supplied to the probes 15, as well as determine to which electrode locations on the probes 15 the actual pulses are sent. As described elsewhere herein, a number of known DBS devices exist and therefore the required stimulation profile and range of parameters are well understood and are fairly standard according to medical practice. The nature of the pulses is determined by the following five parameters: (1) frequency, (2) amplitude, (3) pulse width, (4) on/off state (i.e., whether pulses are generated and/or provided to any electrodes at all), and (5) application location (i.e., to which particular electrodes the pulses are applied). These parameters are illustrated in FIG. 3. Current DBS devices have frequency, amplitude and pulse width ranges of about 2-185 Hz, 0-10.5 V, and 60-450 μs, respectively, although these complete ranges are not fully used. Typically, in DBS, the pulses administered to the brain are between 60 and 240 μs biphasic waveforms with a frequency of about 185 Hz. In addition, the pulses range in amplitude from about 1.5 V to 3 V, although normally the amplitude does not exceed 2.5 V.

In the particular embodiment of the DBS device 5 shown in FIGS. 1 and 2, the probes 15 include four electrodes for providing pulses to any one or any combination of four locations in the brain. In addition, in the particular embodiment of the DBS device 5 shown in FIGS. 1 and 2, the amplitude, frequency and pulse width of the pulses that are provided to the electrodes may be varied (four different amplitudes are possible). It will be appreciated, however, that this embodiment is meant to be exemplary only and that more or less probes and more or less voltage levels may be employed in a device without departing from the scope of the present invention. The actual pulses that are created and to which location or locations (i.e., which probes) they are provided is determined by parameters that, as noted above, are programmed in the processor 65. It is important in any DBS device for these parameters to be selectively adjustable, as the appropriate pulse frequency, amplitude and width must be selected and possibly later adjusted for each individual patient. Thus, the DBS device 5 of the present invention is, as described in greater detail herein, provided with a mechanism for selectively adjusting these parameters.

As stated above, the processor 65 (FIG. 2) creates and outputs pulses according to the selected pulse parameters. The circuitry in control circuitry 45 for adjusting the amplitude of the pulses as desired is realized by voltage dividers 70, which consist of four separate voltage dividers, one for each voltage level. Each voltage divider is driven by a direct pulse from the processor 65. The control circuitry 45 also includes a bank of analog switches 75 and a bank of analog switches 80. The processor 65 sends a signal to the bank of analog switches 75 to close a selected one of the switches to allow the output of a particular voltage divider (the chosen voltage level) to be passed through. In addition, the processor sends a signal to the bank of analog switches 80 to close those switches that are associated with the particular electrodes of probes 15 that are to receive the pulses.

According to an aspect of the present invention, the implantable device 10 is adapted to preserve power when pulsing is not required. Specifically, the processor 65 includes a watchdog timer, and the watchdog timer timeout, used as the wakeup mechanism, can be scaled down so that the processor 65 enters a sleep mode between pulses. In addition, a low power RC oscillator external to the processor 65 may be used with the processor 65 for clocking purposes such that its internal, high speed oscillator can be turned off to further persevere power.

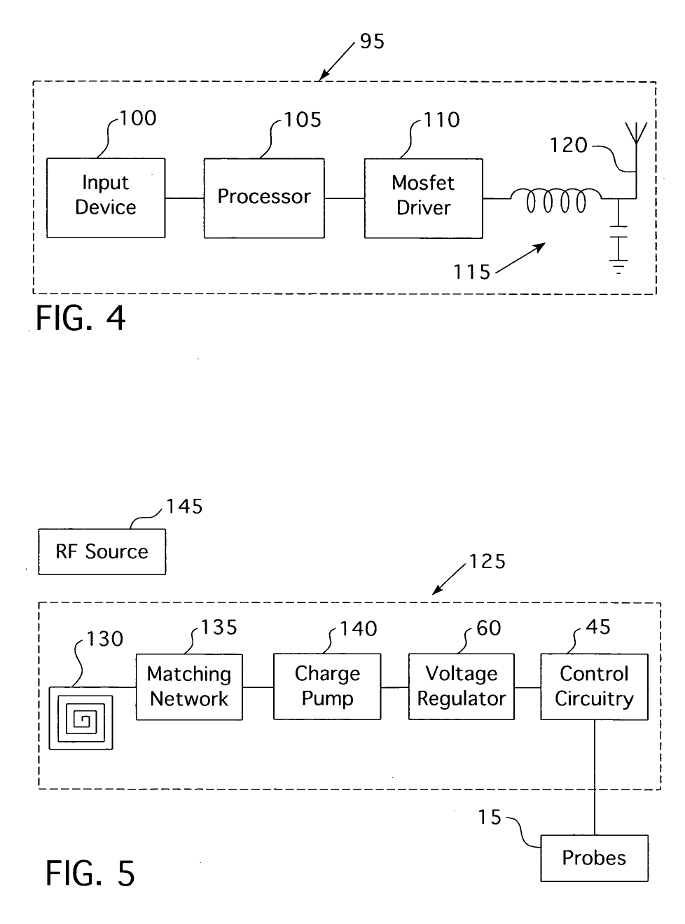

As noted above, it is preferred to be able to selectively adjust the pulsing parameters within the processor 65. Thus, according to a further aspect of the present invention, the DBS device 5 is provided with a mechanism for remotely and wirelessly programming the processor 65 so that the pulse parameters can be selectively adjusted. For this purpose, the control circuitry 45 includes a wireless communications device 85 having an antenna 90 that is in electronic communication with the processor 65 when it is necessary to perform adjustments. The wireless communications device 85 is adapted to receive programming signals sent from a remote programming device 95 shown in block diagram form in FIG. 4 and described hereinafter. The wireless communications device 85 may be any wireless receiver or transceiver that is able to communicate via any of a number of known wireless communications protocols, including, without limitation, an RF protocol such as Bluetooth. A suitable device that may be used for the wireless communications device 85 is the ATA5283 low power receiver that was sold by Atmel Corporation of San Jose, Calif. That particular device uses a simple ASK protocol at a frequency of 125 KHz and stays in a standby (low power sleep) mode until it senses a 125 KHz preamble of at least 5.64 ms, after which it wakes up and outputs digital data based on the presence of the 125 KHz signal. After data transmission, a simple digital high input to the reset pin puts the device back to sleep. The antenna used in this application is a small wire wrapped around the circuitry perimeter.

FIG. 4 is a block diagram of the remote programming device 95 that allows an operator to set pulsing parameters for the DBS device 5 and transmits programming signals which will cause the processor 65 to implement the selected parameters. The remote programming device 95 includes an input device 100 that enables an operator to set desired programming values. The input device 100 may be any suitable mechanism for inputting data, such as, without limitation, a keypad, a touch screen, or a series of slide switches. The input device 100 is in electronic communication with a processor 105 so that the data input by the operator can be sent thereto. The processor 105 is adapted to receive the input signals relating to the desired pulse parameters and convert them into programming signals appropriate for programming the processor 65 of the control circuitry 45. The processor 105 is preferably a microcontroller such as the PIC16LF87 microcontroller sold by Microchip technology, Inc. of Chandler, Ariz. Most suitable processors are not able to create a healthy sinusoid for transmitting the programming signals. As a result, in order to generate a signal appropriate for transmission, the processor 105 sends the programming signal pulses to a MOSFET driver 110, such as the TC4422 driver sold by Microchip corporation, provided as part of the remote programming device 95 which in turn drives an LC circuit 115 also provided as part of the remote programming device 95. The MOSFET driver 110 is powered by a separate 12 V power supply (not shown) so as to provide enough current to drive the high voltage and current oscillations in the LC circuit 115. In addition, the LC circuit 115 alone is not sufficient to send a strong signal to the control circuitry 45 (FIG. 1), but instead employs an antenna 120 to transmit the 125 KHz signal more efficiently. For this purpose, a PhidgetRFID antenna sold by Phidgets Inc, Calgary, Canada, designed for use with 125 KHz RFID systems, may be used for antenna 120. It will be appreciated that other suitable wireless transmitting devices, such as various commercially available transmitter and/or transceiver chips and antennas, may also be used without departing from the scope of the present invention.

FIG. 5 is a block diagram of an implantable DBS device 125 connected to implanted probes 15 according to an alternative embodiment of the present invention. The DBS device 125, like the DBS device 5, is adapted to be implanted subcutaneously in the head. In addition, the DBS device 125 does not have an onboard power supply such as a battery. Instead, the DBS device 125 is powered by harvesting energy that is transmitted in space. A number of methods and apparatus for harvesting energy from space and using the harvested energy to power an electronic device are described in U.S. Pat. No. 6,289,237, entitled “Apparatus for Energizing a Remote Station and Related Method,” U.S. Pat. No. 6,615,074, entitled “Apparatus for Energizing a Remote Station and Related Method,” U.S. Pat. No. 6,856,291, entitled “Energy Harvesting Circuits and Associated Methods,” and U.S. Patent Application Publication No. 2005/0030181, entitled “Antenna on a Wireless Untethered Device such as a Chip or Printed Circuit Board for Harvesting Energy from Space,” each assigned to the assignee hereof, the disclosures of which are incorporated herein by reference.

The DBS device 125 includes an antenna 130, which, in the embodiment shown in FIG. 5, is a square spiral antenna. The antenna 130 is electrically connected to a matching network 135, which in turn is electrically connected to a voltage boosting and rectifying circuit in the form of a charge pump 140. The charge pump 140 is electrically connected to a voltage regulator 60 which is electrically connected to the control circuitry 45. The control circuitry 45 is as described above in connection with FIG. 2 and controls the generation of the electrical pulses provided to the probes 15 (and ultimately to the patient's brain).

In operation, the antenna 130 receives energy, such as RF energy, that is transmitted in space by an RF source 145. The RF source 145 may be, without limitation, a local radio station. The RF energy received by the antenna 130 is provided, in the form of an AC signal, to the charge pump 140 through the matching network 135. The charge pump 140 amplifies and rectifies the received AC signal and provides the resulting DC signal to the voltage regulator 60. The voltage regulator 60 provides a regulated DC signal to the control circuitry 45 as a power supply. Thus, the DBS device 125 is able to be powered remotely without the need for an onboard power supply or energy storage device such as a capacitor or rechargeable battery.

The matching network 135 matches the impedance of the charge pump 140 to the impedance of the antenna 130 as complex conjugates for optimal antenna performance. In one particular embodiment, the matching network is an LC tank circuit formed by the inherent distributed inductance and inherent distributed capacitance of the conducing elements of the antenna 130. Such an LC tank circuit has a non-zero resistance R which results in the retransmission of some of the incident RF energy. This retransmission of energy may cause the effective area of the antenna 130 to be greater than the physical area of the antenna 130.

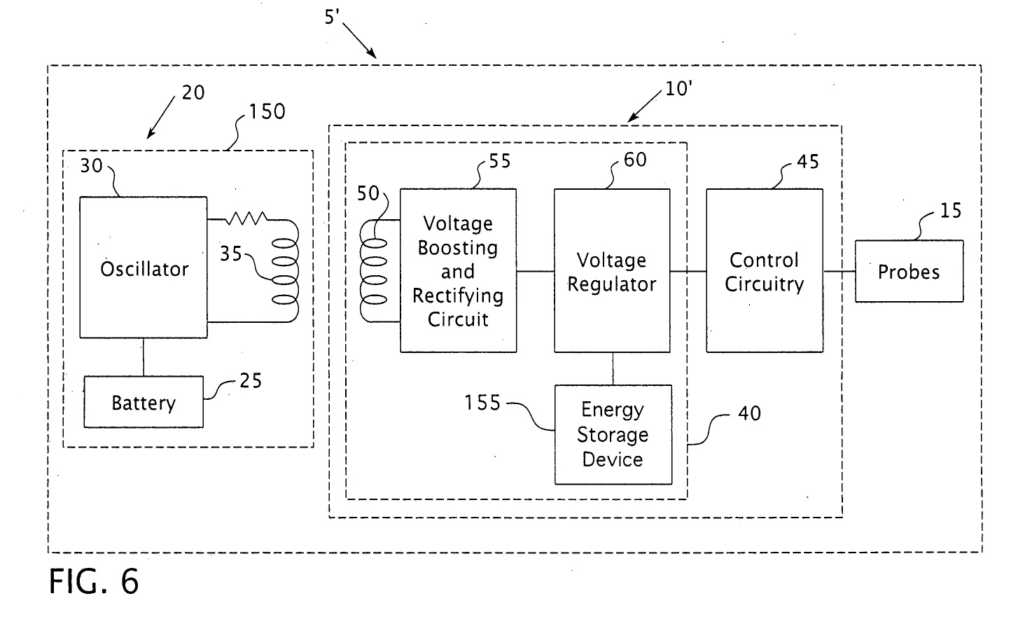

FIG. 6 is a block diagram of a DBS device 5′ according to an alternative embodiment of the present invention that is, except as described below, identical to the DBS device 5 shown in FIG. 1. In the DBS device 5′, the power supply 20 is provided in a stationary location 150, such as within the headboard of the patient's bed. The implantable device 10′ is identical to the implantable device 10 shown in FIG. 1 except that it includes power circuitry 40′ that includes an energy storage device 155, which may be, without limitation, a capacitor such as a so-called super capacitor (on the order of at least 0.2-10 F ) or a rechargeable battery. In operation, the implantable device 10′ receives power from the power supply 20 by near-field inductive coupling in the manner described elsewhere herein when the implantable device 10′ is in proximity with the power supply 20. As used herein, proximity means that the secondary winding 50 is within the field generated by the primary winding 35. In the embodiment where the power supply 20 is provided in the headboard of the patient's bed, the implantable device 10′ will be in proximity with the power supply 20 when the patient is sleeping. The AC signal that is generated by the near-field inductive coupling is amplified and rectified in the manner described in connection with implantable device 10. However, in the case of the implantable device 10′, the resulting DC signal that is generated is used to: (i) power the control circuitry 45, and (ii) charge the energy storage device 155 so that the power that is stored therein may later be used to power the control circuitry 45 when the implantable device 10′ is no longer in proximity with the power supply 20. The operation of the implantable device 10′ is otherwise identical to the operation of the implantable device 10.

While preferred embodiments of the invention have been described and illustrated above, it should be understood that these are exemplary of the invention and are not to be considered as limiting. Additions, deletions, substitutions, and other modifications can be made without departing from the spirit or scope of the present invention. Accordingly, the invention is not to be considered as limited by the foregoing description but is only limited by the scope of the appended claims.

What is claimed is:

1. An apparatus for providing electrical stimulation to the brain of a patient, comprising:
one or more probes for being implanted in said brain and for providing electrical pulses to said brain;
an implantable device for being implanted subcutaneously in said patient's head, said implantable device having: (i) control circuitry electrically connected to said one or more probes, said control circuitry being adapted to generate said electrical pulses and provide said electrical pulses to said one or more probes, and (ii) power circuitry electrically connected to said control circuitry, said power circuitry providing a DC power signal to said control circuitry; and
a power supply provided at a stationary location separate from said implantable device and external to said patient's body, said power supply providing power to said implantable device through a near-field technique between said power supply and said power circuitry when said power circuitry is in proximity with said power supply.

2. The apparatus according to claim 1, wherein said near-field technique is near-field inductive coupling between said power supply and said power circuitry when said power circuitry is in proximity with said power supply.

3. The apparatus according to claim 2, wherein said power supply includes an oscillator and a primary winding, said oscillator generating a first AC signal and providing said first AC signal to said primary winding, wherein said power circuitry includes a secondary winding, wherein said first AC signal induces a second AC signal in said secondary winding when said secondary winding is in proximity with said primary winding, and wherein said power circuitry converts said second AC signal into said DC power signal.

4. The apparatus according to claim 3, wherein said power circuitry includes a voltage boosting and rectifying circuit that converts said second AC signal into a first DC signal, and a voltage regulator that receives said first DC signal and generates said DC power signal based thereon.

5. The apparatus according to claim 4, wherein said voltage boosting and rectifying circuit is a one or more stage charge pump.

6. The apparatus according to claim 1, wherein said control circuitry includes a programmable processor and a wireless communications device, said programmable processor controlling the generation of said electrical pulses based upon one or more pulse parameters, and wherein said apparatus further comprises a remote programming device external to said patient's body, said remote programming device being adapted to wirelessly transmit programming signals to said wireless communications device, said programming signals being provided to said programmable processor for adjusting said one or more pulse parameters.

7. The apparatus according to claim 6, wherein said one or more pulse parameters specify one or more of a frequency, an amplitude, a pulse width, an on/off state, and an application location of said electrical pulses.

8. The apparatus according to claim 1, wherein said power circuitry includes an energy storage device for storing at least a portion of said power for subsequent use by said implantable device.

9. The apparatus according to claim 1, wherein said power supply is provided as part of a piece of furniture.

10. The apparatus according to claim 9, wherein said power supply is provided as part of a bed.

11. A method of providing electrical stimulation to the brain of a patient, comprising:
implanting one or more probes into said brain, said one or more probes being adapted to provide electrical pulses to said brain;
implanting a device subcutaneously in said patient's head, said device being electrically connected to said one or more probes;
causing said device to generate said electrical pulses and provide said electrical pulses to said one or more probes; and
providing power to said device from a stationary location external to said patient's body using a near-field technique.

12. The method according to claim 11, wherein said near-field technique is near-field inductive coupling.

13. The method according to claim 12, wherein said step of providing power includes generating a first AC signal at said location external to said patient's body, said first AC signal inducing a second AC signal in said device, and converting said second AC signal to a DC power signal for powering said device.

14. The method according to claim 11, wherein said electrical pulses are generated based upon one or more pulse parameters, the method further comprising selectively wirelessly adjusting said one or more pulse parameters from a second location external to said patient's body.

15. The method according to claim 14, wherein said one or more pulse parameters specify one or more of a frequency, an amplitude, a pulse width, an on/off state, and an application location of said electrical pulses.

16. The method according to claim 11, further comprising storing at least a portion of said power for subsequent use by said device.

17. The method according to claim 16, wherein the stored power is used by said device when said device is located more than a certain distance from said stationary location.

18. The method according to claim 11, wherein said stationary location comprises a piece of furniture.

19. The method according to claim 18, wherein said piece of furniture is a bed.

20. A method of treating a neurodegenerative disease, comprising:
implanting a device in the head of a patient;
causing said device to generate and provide electrical pulses to said brain; and
providing power to said device from a stationary location external to said patient's body using a near-field technique.

21. The method according to claim 20, wherein said near-field technique is near-field inductive coupling.

22. The method according to claim 21, wherein said step of providing power includes generating a first AC signal at said location external to said patient's body, said first AC signal inducing a second AC signal in said device, and converting said second AC signal to a DC power signal for powering said device.

23. The method according to claim 20, wherein said electrical pulses are generated based upon one or more pulse parameters, the method further comprising selectively wirelessly adjusting said one or more pulse parameters from a second location external to said patient's body.

24. The method according to claim 23, wherein said one or more pulse parameters specify one or more of a frequency, an amplitude, a pulse width, an on/off state, and an application location of said electrical pulses.

25. The method according to claim 20, further comprising storing at least a portion of said power for subsequent use by said device.

26. The method according to claim 25, wherein the stored power is used by said device when said device is located more than a certain distance from said stationary location.

27. The method according to claim 20, wherein said stationary location comprises a piece of furniture.

28. The method according to claim 27, wherein said piece of furniture is a bed.
